(19) United States
(12) Patent Application Publication
(10) Pub. No.: US 2023/0015942 A1
(43) Pub. Date: Jan. 19, 2023

(54) MICRONEEDLE DELIVERY DEVICE WITH DETACHABLE HYBRID MICRONEEDLE DEPOTS FOR LOCALIZED DELIVERY OF CELLS

(71) Applicant: THE REGENTS OF THE UNIVERSITY OF CALIFORNIA, Oakland, CA (US)

(72) Inventors: KangJu Lee, Los Angeles, CA (US); Alireza Khademhosseini, Los Angeles, CA (US)

(73) Assignee: THE REGENTS OF THE UNIVERSITY OF CALIFORNIA, Oakland, CA (US)

(21) Appl. No.: 17/783,265

(22) PCT Filed: Dec. 10, 2020
(86) PCT No.: PCT/US2020/064336

§ 371 Date: Jun. 7, 2022

Related U.S. Application Data
(60) Provisional application No. 62/946,709, filed on Dec. 11, 2019.

(51) Int. Cl. A61M 37/00; A61K 35/28; A61K 47/10; A61K 47/32; A61K 47/36

(52) U.S. Cl. CPC A61M 37/0015; A61K 35/28; A61K 47/10; A61K 47/32; A61K 47/36; A61M 2037/0046

(57) ABSTRACT

A delivery device or patch is disclosed that includes a detachable hybrid microneedle depot (d-HMND) for cell delivery. The system includes, in one embodiment, an array of microneedles formed from an outer PLGA shell and an internal gelatin methacryloyl (GelMA)-mesenchymal stem cells (MSC) mixture (GMM). The array of microneedles project from a base substrate layer that may be flexible. The therapeutic device may be applied to a tissue site of interest and the base substrate layer is removed leaving the hybrid microneedles in the tissue at the site of application to deliver MSCs. Other stem/therapeutic cells may also be delivered with the hybrid microneedles.

### RELATED APPLICATION

This application claims priority to U.S. Provisional Patent Application No. 62/946,709 filed on Dec. 11, 2019, which is hereby incorporated by reference in its entirety. Priority is claimed pursuant to 35 U.S.C. § 119 and any other applicable statute.

### STATEMENT REGARDING FEDERALLY SPONSORED RESEARCH AND DEVELOPMENT

This invention was made with government support under Grant Number GM126831, awarded by the National Institutes of Health. The government has certain rights in the invention.

### TECHNICAL FIELD

The technical field generally relates to biocompatible microneedles. More particularly, the technical field relates to a delivery device (e.g., patch) that incorporates detachable microneedles for the delivery of cells (e.g., stem cells). The detachable microneedles are hybrid structures that include an outer shell and an inner crosslinked gelatin material that contains the cells.

### BACKGROUND

The application of stem cell biology to tissue regeneration has undergone a remarkable evolution and has generated great interest due to its potent ability to form, repair, and maintain tissues and organs after injury. Stem cells can not only differentiate into different functional cells which can promote the overall process of regeneration, but also can regenerate damaged tissue through the secretion of functional growth factors that stimulate and promote tissue regeneration by angiogenesis, remodeling, cellular recruitment and immune modulation. Among the several types of stem cells, mesenchymal stem cells (MSCs) have demonstrated the most clinical promise for treating tissue damage because of their wide tissue distribution, ease of isolation, compatibility with ex vivo culture and their immunosuppressive abilities. Thus, MSCs have been intensively studied in preclinical and clinical studies for regenerative applications and treating inflammation caused by cardiovascular disease, myocardial infarction, brain and spinal cord injury, bone and cartilage injuries and liver fibrosis.

Historically, MSCs have been administrated by injection into blood vessels. Bone marrow-derived MSCs could be used to treat cardiac damage by catheter-based trans-endocardial injections. Chronically infarcted myocardium has been regenerated via long-term engraftment and trilineage differentiation of MSCs. Multiple MSC transplantations were made safe and effective by intrathecal injection. Other delivery methods include intravenous and intranasal injection of MSCs for brain injuries, intra-articular injection of autologous MSCs for knee osteoarthritis, and intrasplenic injection of exosomes secreted by MSCs for liver injuries. However, achieving a therapeutic response requires the delivery of a massive number of stem cells to a specific site with high precision. Therefore, clinical translation of MSC therapeutics will be difficult to achieve due to low engraftment efficiency. As a result, significant effort has been devoted to increasing the efficiency and stability of MSC delivery by combining metallic, polymeric, and hydrogel-based microparticles. These approaches enhance the reparative potential of conventional MSCs and promote proper cellular function which improves cryopreservation and lyophilization stability. Although advanced local transplantation or injection of MSCs shows some regenerative potential, these methods still require excessive cell production and in situ injection to the target tissue may cause adverse effects or further damage to the tissue. Despite these risks, direct injection of MSCs to the lesion, particularly for myocardial infarction (MI), have been introduced and are the leading clinical practice. These procedures are invasive and have a risk of forming fibrotic scar tissue.

Various attempts have also been made to improve localization of the cells once delivered. MSCs have been embedded in scaffolds consisting of a wide range of materials. The entrapment of MSCs in thermally expandable hydrogel patches has been demonstrated to promote cell adhesion and spreading activity. In addition, a polysaccharide-incorporated silk fibroin, chitosan, and hyaluronic acid-hybrid patch was designed to enhance the proliferation and cardiomyogenic differentiations of MSCs. This hybrid patch with embedded MSCs was implanted into a rat MI model. See Chi et al., Cardiac repair achieved by bone marrow mesenchymal stem cells/silk fibroin/hyaluronic acid patches in a rat of myocardial infarction model, Biomaterials, 2012 August; 33(22):5541-51 doi: 10.1016/j.biomaterials.2012.04.030. Epub 2012 May 8. A commercial product, CardioCel®, made from processed bovine pericardium has been manufactured and embedded with MSCs as well. CardioCel® apparently showed good therapeutic efficacy for cardiovascular cell therapy. See Vashi et al., Evaluation of an established pericardium patch for delivery of mesenchymal stem cells to cardiac tissue, Journal of Biomedical Materials research, Part A, 29 Sep. 2014, 103(6):1999-2005 DOI: 10.1002/jbm.a.35335. However, dynamic tissue barriers prevent minimally invasive, effective implementation of these therapies. The inability to target these cells to tissues of interest with high efficiency and engraftment has inhibited widespread clinical adoption. Additionally, these MSC-embedded scaffolds usually exhibit low strength of adhesion due to poor cohesive properties which also decreases the cell migration efficiency into injury sites and reduces tissue remodeling efficacy.

To address these challenges, straightforward delivery with minimal tissue damage is required for successful therapeutic application beyond conventional intrastromal/venous injections and patches. Microneedles (MNs) have been shown to be an effective drug delivery vehicle while also minimizing the dose required through localization. Furthermore, recent MN research for vascular or ocular tissues have demonstrated their spatial precision for drug delivery while overcoming the complex and dynamic barriers of the body including multilayered vascular structures or tear turnover and eye blinking. However, conventional molding processes for the fabrication of MNs are not compatible with maintaining cell viability.

### SUMMARY

In one embodiment, a detachable hybrid microneedle depot (d-HMND) delivery device or patch is disclosed for delivering MSCs to a variety of tissues and organs for tissue regeneration (FIGS. 1A-1F). The d-HMND delivery device or patch is made up of gelatin methacryloyl (GelMA) and poly(lactic-co-glycolic) acid (PLGA), both of which are biocompatible and biodegradable. The MSCs are located in a GelMA matrix which improves cell viability after delivery by providing a biologically relevant extracellular matrix (ECM) containing peptides for cell adhesion and matrix degradation and remodeling. In addition, the PLGA shell protects the GelMA-MSC Mixture (GMM) before insertion into the targeted tissue. The solid PLGA shell facilitates facile insertion of the microneedles into the wound bed (or other tissue) with minimal collateral tissue damage. The d-HMND delivery device or patch is designed so that the base substrate layer that holds the microneedles is separated after application so that no foreign materials remain at the site of the wound long-term. In experimental studies, the d-HMND delivery device or patch design was optimized along with fabrication methods. Further, the GelMA properties were characterized for the enhancement of MSC viability. The regenerative efficacy of the d-HMND delivery device or patch was tested using a mouse skin wound model.

In one embodiment, a delivery device for the localized delivery of cells to living tissue is disclosed. The cells, in some embodiments may include stem cells or therapeutic cells. The delivery device includes a base substrate layer having a plurality of microneedles extending away from the surface of the base substrate layer, wherein the plurality of microneedles are formed from an outer hardened shell comprising a biodegradable or dissolvable polymer and an inner region containing a cytocompatible hydrogel mixed with cells therein, and wherein the plurality of microneedles are detachable from the base substrate layer.

In another embodiment, a method of using the delivery device includes placing the delivery device on living tissue of a mammal such that the plurality of microneedles penetrates into the tissue and removing the base substrate layer from contact with the tissue, wherein the plurality of microneedles separates from the base substrate layer and remain in the tissue.

In another embodiment, a method of treating tissue includes inserting a plurality of microneedles into tissue, wherein the plurality of microneedles are formed from an outer hardened shell comprising a biodegradable or dissolvable polymer and an inner region containing a cytocompatible hydrogel-based material mixed with the cells. In one embodiment, the plurality of microneedles are temporarily adhered to a base substrate layer that detaches from the plurality of microneedles and is removed after insertion of the plurality of microneedles into the tissue (with the microneedles remaining in the tissue).

In another embodiment, a method of manufacturing a delivery device for the localized delivery of cells to living tissue includes: providing a mold containing a plurality of needle-shaped cavities therein; applying a solution containing the biodegradable or dissolvable polymer (e.g., poly(lactic-co-glycolic) acid (PLGA)) in a solvent on the mold allowing the solvent to evaporate to form shells in the needle shaped cavities; applying a solution of the cytocompatible hydrogel and cells and a photoinitiator on the mold containing the shells; irradiating the mold containing the solution with light to crosslink the hydrogel to form the plurality of microneedles; applying a base substrate layer having an adhesive thereon to the mold; and removing the base substrate layer from the mold with the plurality of microneedles adhered thereto.

### BRIEF DESCRIPTION OF THE DRAWINGS

FIG. 1A schematically illustrates an assembled d-HMND delivery device or patch that includes of an array of MNs with PLGA shells, filled with a GelMA-MSC mixture, and fixed to a flexible base substrate layer.

FIG. 1B illustrates a magnified view of a single microneedle that is part of the delivery device.

FIG. 1C schematically illustrates the delivery device or patch being applied to tissue to insert the microneedles into the tissue.

FIG. 1D schematically illustrates the working mechanism of the microneedles of the delivery device or patch after application to target tissue. This includes microneedle detachment, shell degradation, and MSC release from the GelMA.

FIG. 1E illustrates a cross-sectional view of a delivery device or patch according to one embodiment.

FIG. 1F illustrates cross-sectional views of a single microneedle attached to the base substrate layer with an adhesive material.

FIG. 2A illustrates a sequence of operations used to prepare GelMA+MSC mixture (GMM).

FIG. 2B illustrates multiple casting steps for PLGA shell fabrication according to one embodiment.

FIG. 2C illustrates the operation of loading GMM into the PLGA shells and subsequent crosslinking operation by exposure to UV radiation.

FIG. 2D illustrates the assembly of a d-HMND delivery device or patch using double-sided tape (base substrate layer in this particular embodiment).

FIG. 3A illustrates a bar graph of MSC viability within 3D GMM structure with respect to different compressive moduli of GelMA modulated by crosslinking time.

FIG. 3B illustrates representative images of MSC viability from live & dead assay at different time points after initiation of crosslinking. Top images showing poor viability in GelMA that is too soft (2 min crosslinking) and too stiff (5 min crosslinking). Bottom images showing excellent viability of MSCs in GelMA with compressive moduli around 20˜30 kPa. (scale bar=100 μm).

FIG. 4A illustrates confocal microscopic images of PLGA shell after the 1^{st} and 2^{nd} molding processes. Left two images are axial cross-sections and the right images are top-viewed cross-sections along the length of the needle (i˜vi). (Scale bars=200 μm).

FIG. 4B illustrates magnified image of the d-HMND delivery device or patch loaded with GMM inside the red-dyed PLGA shell. (scale bar=3 mm).

FIG. 5A illustrates histograms showing the results of a compression test of d-HMND devices containing variably cross-linked GMMs. The inset shows the definition of failure force on graphed on the y-axis.

FIG. 5B is a graph illustrating the mechanical behavior of d-HMND device against compressive deformation.

FIG. 5C is an image of cryosectioned mouse skin after applying the d-HMND device. The inset is a top-view image after d-HMND delivery device or patch application. (Scale bars=500 μm, 3 mm (inset)). *p<0.01, compared with PLGA shell only group. All data are presented as the mean±SD.

FIG. 6A illustrates representative images of MSC viability at time points of 1, 24 and 48 hrs at top, medium, and bottom locations within the microneedle. (Scale bars=100 μm).

FIG. 6B illustrates a graph of time-dependent MSC viability.

FIG. 6C illustrates a histogram illustrating the quantification of VEGF concentration secreted by MSCs cultured over 7 days in two GMMs—one made and used immediately (left histogram of pair, 0 hr), the other made and stored for 24 hours (right histogram of pair, 24 hrs).

FIG. 6D illustrates a graph of accumulated VEGF concentrations from FIG. 6C. All data are presented as the mean±SD.

FIG. 7A illustrates images of wound healing in different experimental groups: untreated (control), MSC-injected, d-HMND delivery device or patch without MSCs, and d-HMND delivery device or patch with MSCs. (Scale bars=10 mm).

FIG. 7B illustrates a graph showing the wound area after 1 and 2 weeks.

FIG. 7C illustrates representative histologic images of wound bed treated by each method at days 7 and 14. (Scale bars=1 mm).

FIG. 7D illustrates a histogram showing re-epithelialization after 2 weeks.

FIG. 7E illustrates a histogram showing MET length after 2 weeks. All data are presented as the mean±SD.

FIG. 8 illustrates images showing the viability of MSC within GMM with respect to the different crosslinking time from 2 to 8 minutes using 14 mV/cm² of UV. (scale bar=100 μm).

FIG. 9A illustrates representative images of compression test of GMM disk in response to increased compression forces (scale bar=10 mm).

FIG. 9B illustrates a bar graph showing compressive Young's modulus of GMM disks.

### DETAILED DESCRIPTION OF ILLUSTRATED EMBODIMENTS

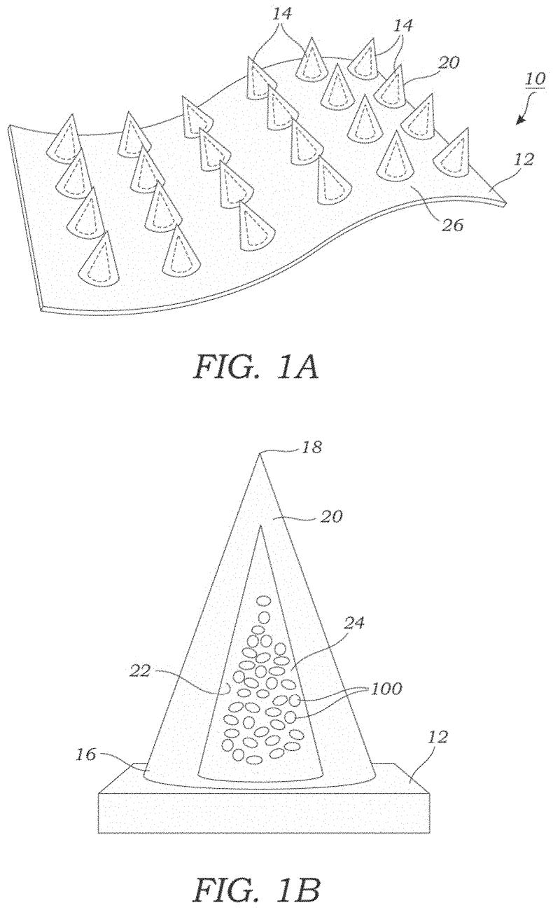

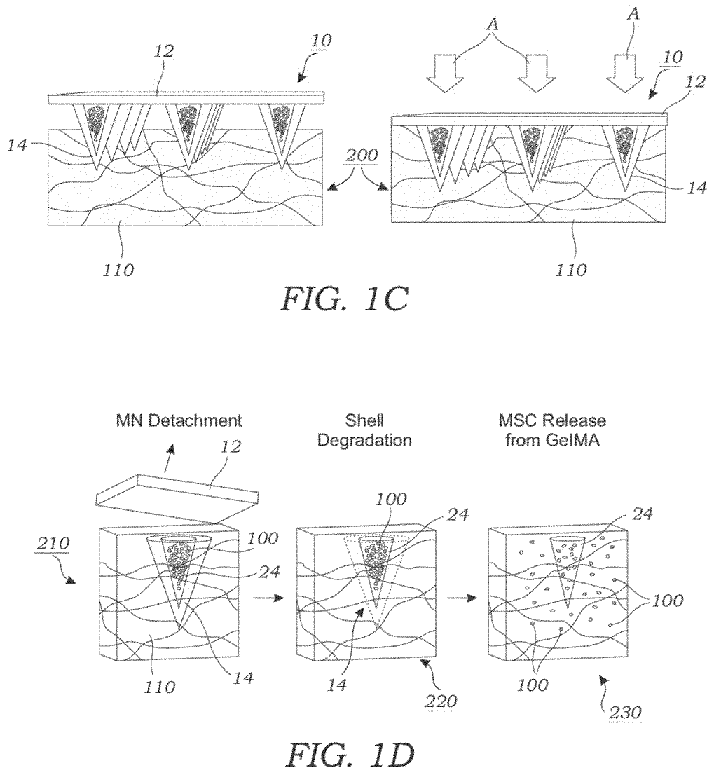

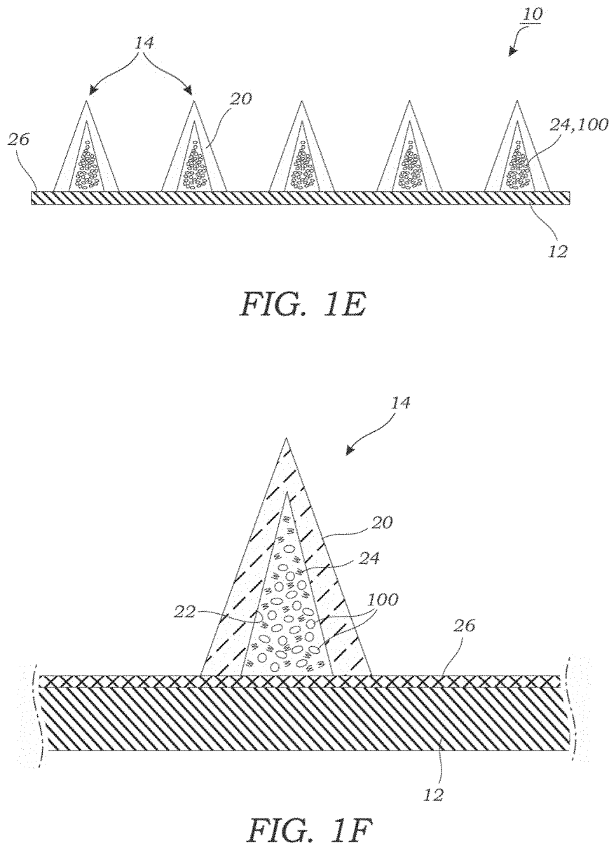

FIGS. 1A-1F illustrate an embodiment of a delivery device 10 (and aspects thereof) for the localized delivery of cells 100 (FIGS. 1B, 1D, 1F) to living tissue 110. The cells 100 that are delivered to tissue 110 are living cells. In one embodiment, the cells 100 are stem cells. An example of such stem cells 100 include mesenchymal stem cells (MSCs). However, other therapeutic/stem cells 100 may also be delivered to tissue 110. These cells 100 include, by way of illustration and not limitation, bronchioalveolar stem cells, bulge epithelial stem cells, corneal epithelial stem cells, cardiac stem cells, epidermal neural crest stem cells, embryonic stem cells, endothelial progenitor cells, hepatic oval cells, hematopoietic stem cells, keratinocyte stem cells, neuronal stem cells, pancreatic stem cells, retinal stem cells.

The delivery device 10 is preferably in the form of a patch 10 that, as explained herein, includes a base substrate layer 12 that acts as a temporary backing that is used to deliver any array of cell-containing microneedles 14 to tissue 110. The actual portion of the delivered aspect of the delivery device 10 is also referred to, in some instances, as a detachable hybrid microneedle depot (d-HMND). The detachable microneedles 14 remain in the tissue 110 while the base substrate layer 12 is removed after application. Thus, the delivery device 10 may be in the form of a patch that has the patch backing (i.e., the base substrate layer 12) removed after the plurality of microneedles 14 have penetrated the tissue 110. The living tissue 110 may include any type of mammalian tissue. In one particular embodiment, the tissue 110 is skin tissue, however, the patch 10 may be applied to different tissue types 110.

The delivery device or patch 10 is formed with a base substrate layer 12 that has a plurality of microneedles 14 (sometimes referred to herein as MNs) that extend away from the surface of the base substrate layer 12. The microneedles 14 extend generally orthogonal to the base substrate layer 12 when the base substrate layer 12 is in a flat orientation. Each microneedle 14 includes a base 16 and a tip 18. The microneedles 14 are formed from an outer hardened shell 20 that is made from a biodegradable or dissolvable polymer material. Examples of the polymer material for the hardened shell 20 include, by way of example, poly(lactic-co-glycolic) acid (PLGA), polyvinylpyrrolidone (PVP), polyvinyl alcohol (PVA), polycaprolactone (PCL), chitosan, polyhydroxyalkanoates (PHA), polyanhydrides, polyvalerolactone, polydioxanone, polyurethane (PUR), or polyphosphazenes. Inside the outer hardened shell 20 is located an inner region 22 (FIGS. 1A and 1F) that contains a cytocompatible hydrogel-based material 24 that is mixed with the cells 100. The inner region 22 is thus a pocket or void area that can be filled with the hydrogel-based material 24. Examples of cytocompatible hydrogel-based materials 24 include crosslinked gelatin methacryloyl (GelMA) hydrogel, silk fibroin hydrogel, collagen-based hydrogel, alginate-based hydrogel, hyaluronic acid-based hydrogel, cellulose-based hydrogel, or poly(ethylene glycol) (PEG)-based hydrogel.

In one particular embodiment, the outer hardened shell 20 is made from poly(lactic-co-glycolic) acid (PLGA) and the inner region 22 contains crosslinked gelatin methacryloyl (GelMA) 24 mixed with MSCs 100 (or other stem/therapeutic cells) therein (i.e., the hybrid structure of the microneedle 14). In one preferred embodiment, the plurality of the microneedles 14 are detachable from the base substrate layer 12. The microneedles 14 detach from the base substrate layer 12 at the interface between the base 16 of the microneedles 14 and the base substrate layer 12 that forms the patch 10. In some instances, some microneedles 14 may inadvertently remain adhered to the base substrate layer 12 but that majority of microneedles will detach from the base substrate layer 12 and remain in the tissue 110. The plurality of microneedles 14 may be temporarily adhered to the base substrate layer 12 using an adhesive or glue 26 (FIGS. 1A, 1E). The adhesive or glue 26 may be integrated into the base substrate layer 12 such as a tape or the adhesive or glue 26 may be deposited on the base substrate layer 12. Alternatively, the microneedles 14 may be secured to the base substrate layer 12 through a weak bonding layer (e.g., sacrificial layer) that is formed between the microneedle base 16 and the base substrate layer 12.

During use of the device or patch 10, the plurality of microneedles 14 are adhered to the base substrate layer 12 and penetrate the tissue 110 when the device or patch 10 is applied to tissue 110. As seen in operation 200 FIG. 1C, force (represented by arrows A) is applied to the base substrate layer 12 to insert the microneedles 14 into the tissue 110. The base substrate layer 12 is then removed leaving the plurality of microneedles 14 disposed within the tissue 110. This is illustrated as operation 210 in FIG. 1D. The hardened PLGA shell 20 of the microneedles 14 then undergoes degradation or dissolves (operation 220 in FIG. 1D) and releases the MSCs 100 (or other cells 100) from the GelMA (operation 230 in FIG. 1D). The detachable microneedles 14 thus act as a depot or reservoir for the localized delivery of stem cells 100 to tissue 110.

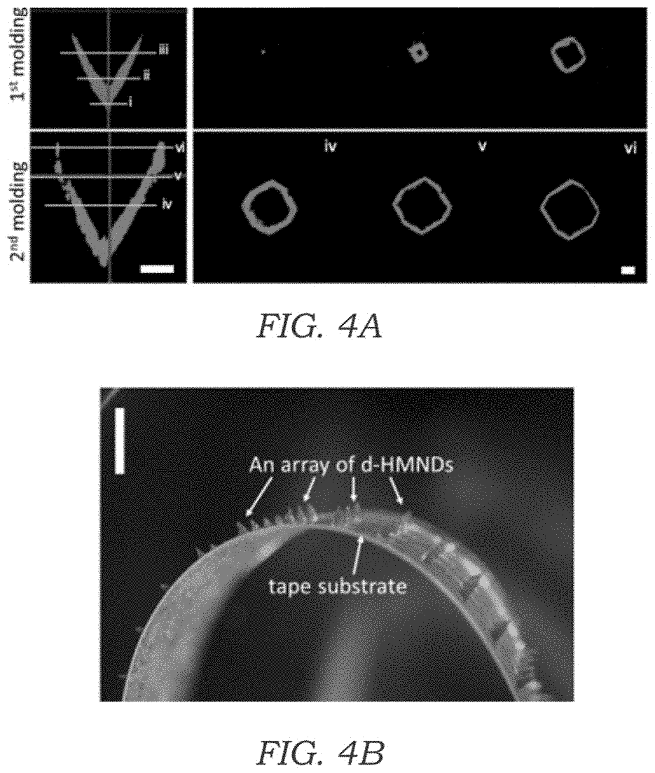

The microneedles 14 have sharpened tips 18 that aid in penetrating tissue 110. The length of the microneedles 14 is typically less than about 1.5 mm and typically fall within the range of about 10 μm and 1,500 μm. More typically, the microneedles 14 have a length of several hundred micrometers (e.g., 700 μm). The height of the microneedles 14 is larger than the width or diameter of the base 16 (e.g., aspect ratio of around 1.5). The pitch or density of the microneedles 14 on the base substrate layer 12 may vary. In one embodiment, there is a center-to-center spacing of around 1,850 μm, although a wide variety of densities may be used. The base substrate layer 12 is preferably flexible in one embodiment (e.g., FIG. 4B) and may be in the form of a tape or the like that can conform to the surface of the tissue 110 on which it is applied. In one embodiment, the crosslinked gelatin methacryloyl (GelMA) mixed with MSCs 100 may have a stiffness between about 10 and about 50 kPa and in a more preferred embodiment, between about 25 and about 35 kPa.

In another embodiment, a method of manufacturing a patch 10 for the localized delivery of stem cells 100 (e.g., MSCs) to living tissue 110 includes providing a mold 40 containing a plurality of needle-shaped cavities 42 therein. A solution containing poly(lactic-co-glycolic) acid (PLGA) in a solvent is applied on the mold 40 allowing the solvent to evaporate to form shells 20 in the needle shaped cavities 42. Multiple rounds of this may be needed to generate the final shell 20 structure. Next, a solution of gelatin methacryloyl (GelMA) 24 (or other hydrogel material 24), stem cells 100, and a photoinitiator is applied on the mold 40 containing the shells 20. The mold 40 containing the solution is then irradiated with light to crosslink the GelMA 24 to form the plurality of microneedles 14. A base substrate layer 12 having an adhesive 26 thereon is then applied to the mold 40. The base substrate layer 12 is then removed from the mold 40 with the plurality of microneedles 14 adhered thereto to form the completed patch 10. As an alternative to light-initiated crosslinking, in other embodiments, the hydrogel material may be crosslinked by exposure to a crosslinking agent.

### EXPERIMENTAL

Characterization of GelMA-MSC Mixture for Cell Viability

GelMA hydrogel material 24 was synthesized to compose the needle-filling scaffold housing the MSCs 100 within the PLGA MN shell 20. GelMA 24 was prepared according to previously reported protocols. See Z. Luo et al., Biodegradable Gelatin Methacryloyl Microneedles for Transdermal Drug Delivery, Advanced healthcare materials 2019, 8, 1801054. Briefly, a 10% (w/v) gelatin solution was made by dissolving gelatin powder in 100 mL phosphate-buffered saline (PBS) and then 8 mL of methacrylic anhydride was added before an additional 100 mL of PBS was added. Subsequently, the solution was dialyzed to remove impurities. The purified solution was filtered and frozen to −80° C. and subsequently lyophilized. Mesenchymal stem cells 100 were cultured in Dulbecco's Modified Eagles Medium-low glucose (DMEM-LG) supplemented with 10% fetal bovine serum (FBS) and 1% Penicillin Streptomycin (PS). MSCs (passages 3-7) from ATCC were used for the experiments. To load the GMM into the shell 20 of the microneedles 14, 10% freeze dried GelMA and 0.5% photoinitiator (e.g., Irgacure 2959) were dissolved into MSC media (w/v). The isolated MSCs 100 were then mixed with prepolymer solution at a cell density of 10⁷ cells/mL.

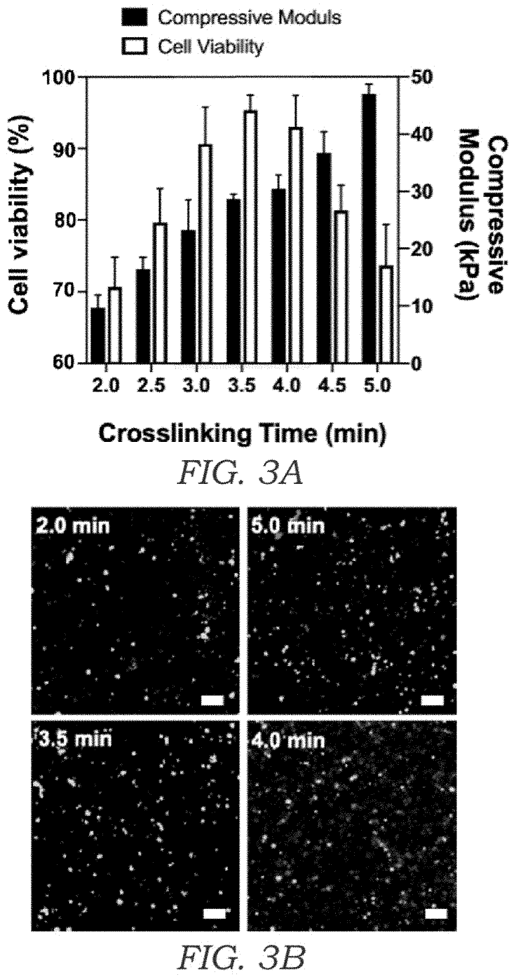

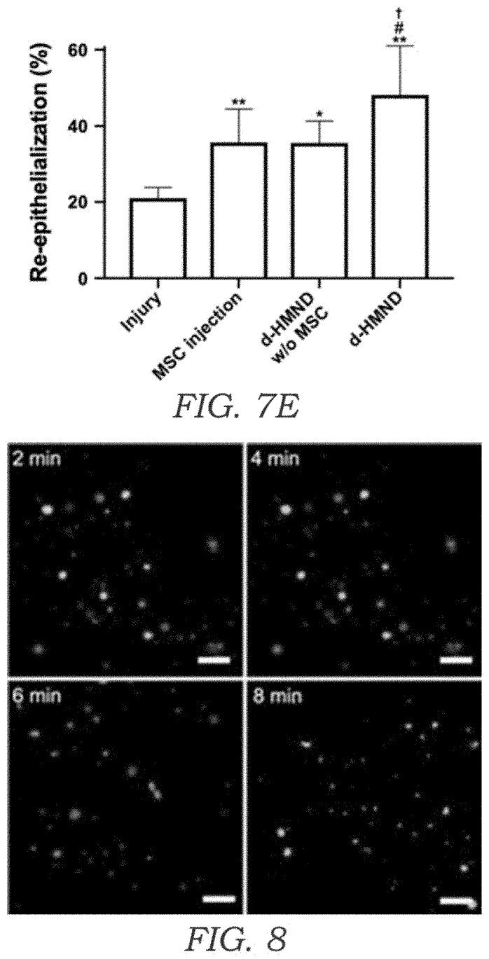

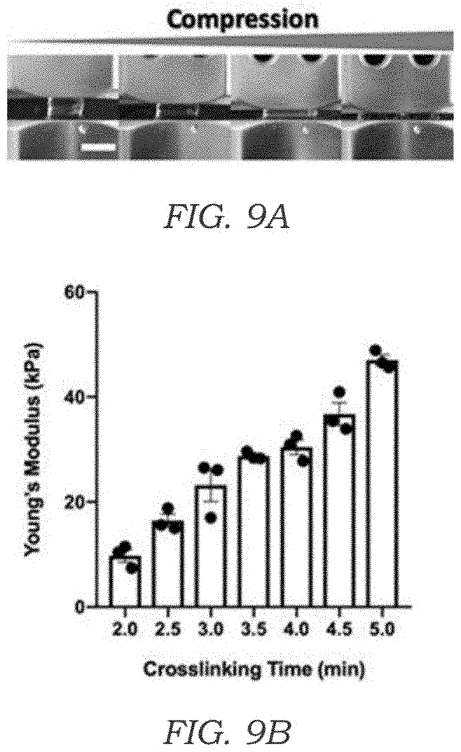

MSC phenotype and differentiation is highly dependent on the properties of the surrounding environment including the mechanical properties and material patterns. In order to optimize cellular behavior to maximize regenerative capacity, the mechanical properties of GelMA 24 were first manipulated by varying the crosslinking time between 2 and 5 minutes. To analyze the mechanical properties of the gel, uniform disks (2 mm height×8 mm diameter) of GMM were crosslinked. A light intensity of 14 mV/cm² was used to crosslink the GMM; this intensity over the required amount of time did not significantly impact cell viability. MSC viability exceeded 90% up to 6 minutes of crosslinking (FIG. 8). After crosslinking with different light intensities, the compressive moduli of GMM disks were measured using a universal testing system (FIGS. 9A, 9B). As shown in FIG. 3A (left bar), the stiffness of the GMM disk can be tuned between 10 and 50 kPa by increasing crosslinking time. To evaluate the influence of stiffness on the viability of encapsulated MSCs 100, an array of GMM disks with different stiffnesses were made and stored in cell media for 24 hours. Subsequently, the viability of MSCs 100 within the GMM disks was analyzed using a live/dead cell assay. It was found that the cell viability was over 90% when MSCs 100 were encapsulated in materials with a stiffness between 25 and 35 kPa and a crosslinking time around 3 to 4 minutes (FIG. 3A). Both overly soft and stiff environments (both top images of FIG. 3B) led to poor cell viability, indicating that there is an ideal range of stiffnesses that maximize MSC growth.

GelMA is one of the biomaterials that has been vigorously studied and used for bioengineering and biomedical applications. Since GelMA has the ability to interact with cells, it is advantageous for three-dimensional (3D) cell culture by acting as ECM. A variety of manufacturing methods have been used to create GelMA scaffolds for cell growth including layer-by-layer stacking, micromolding or patterning, and 3D printing. GelMA, in each individual study, has been characterized and optimized for the specific cell types and tissues used. Here, one of the main parameters to be optimized was the stiffness of the GelMA hydrogel 24 surrounding the MSCs 100. It was also found that cell media was an effective solvent for the creation of GelMA solutions as it facilitated the supply of nutrition to the MSCs in a closed system. Finally, it was demonstrated that a compressive modulus of approximately 30 kPa maximized MSC viability in this system.

Fabrication of Delivery Devices or Patches with Detachable Hybrid Microneedle Depots (d-HMNDs)

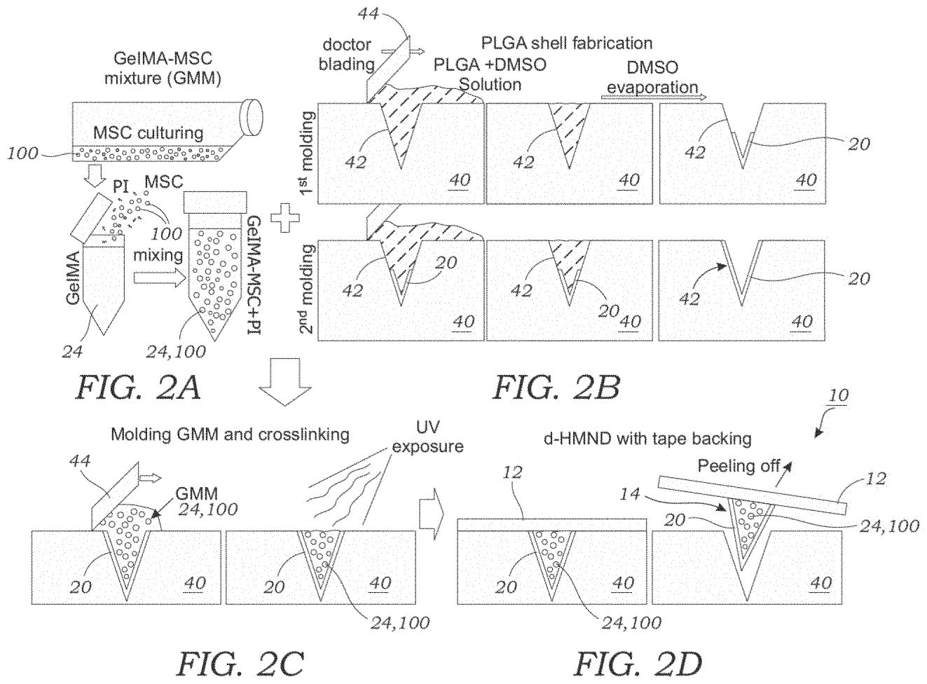

A male MN array, with microneedles 14 having a length of 700 μm, 1850 μm center-to-center spacing, and 1.5 aspect ratio, was fabricated as a master mold using lithography, multiple layer deposition, and wet etching processes. Several process parameters were modified including the size of the square micropattern from previous fabrication process. Next, a polydimethylsiloxane (PDMS) female MN cavity was molded over the microfabricated MN array. After curing, the solidified PDMS was removed from the master mold to yield the MN cavities 42. To form the PLGA shell 20 a mixture of PLGA and dimethyl sulfoxide (DMSO) (PLGA:DMSO=1:3, w:w) were cast into the mold 40 (FIG. 2B). Prior to casting, the PDMS was treated with O₂ plasma to yield a hydrophilic surface allowing the PLGA solution to fill the MN cavities 42 and better adhere to the walls of mold 40. Excess PLGA was removed by doctor blading using blade 44 (e.g., blade as illustrated in FIG. 2C), ensuring that the same amount of PLGA solution was used. The DMSO was evaporated from the solution in the MN cavity 42 to deposit the PLGA along the walls and solidify the shell 20. To achieve the desired final PLGA shell characteristics and uniformity, the casting process was repeated, casting additional PLGA solution over the previously casted shells 20 (FIG. 2B illustrates two such molding cycles). To investigate the formation of the PLGA shell 20, the PLGA solution was spiked with rhodamine B (RB), a visualizing agent, and imaged using confocal microscopy after the first and second molding. Each successive casting step deposited additional material to form the MN shell 20 (FIG. 4A). At each confocal cross-section FIG. 4A), the PLGA shell lined the wall. After a single casting step, the thickness of the PLGA shell 20 at the tip 18 (i) was greater than that of the middle layer (iii). However, after two casting steps the overall thickness of the PLGA shell 20 from the tip 18 (i) to the top layer (vi) was distributed uniformly formed to mimic the profile of the wall (iv-vi, FIG. 4A).

Next, as described in FIG. 2C, the PLGA shell 20 was filled with GMM (hydrogel material 24 and cells 100), removed the excess material using blade 44 to yield a flat surface, and crosslinked the GMM within the shell 20. To better release the MNs 14 from the mold 40, the PDMS mold 40 was manually stretched to form gaps at the interface between the PLGA shell and PDMS surface. Then, commercial double-sided tape was used as the base substrate layer 12 and was attached to the top of the PDMS mold 40 and peeled off (as seen in FIG. 2D) to yield the final patch 10 (FIG. 4B).

Mechanical Property and Tissue Insertion of Microneedles (d-HMND)

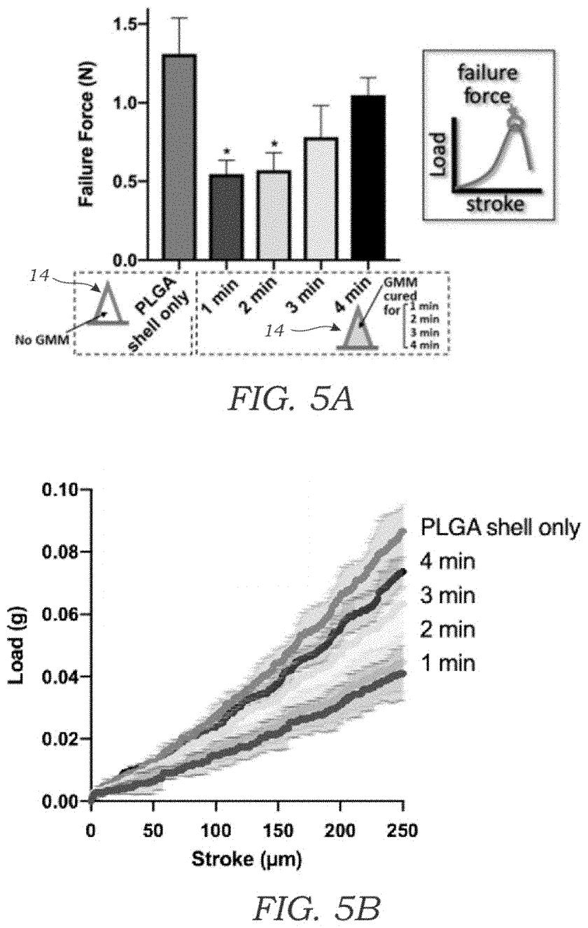

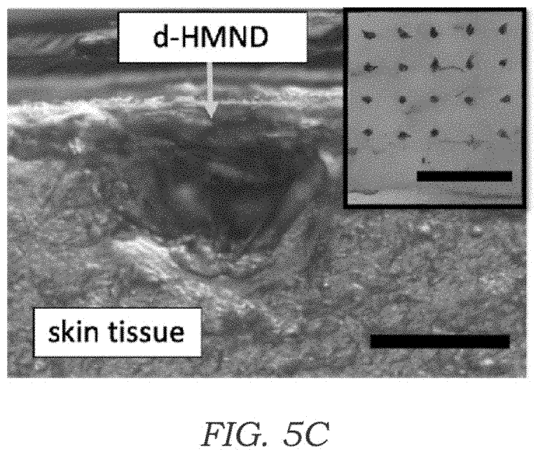

Once the manufacturing process was complete, material stability was a concern. The moisture from the GMM inside the shell 20 can partially degrade the PLGA and diminish the structural integrity of the MN 14. For this reason, the effect of moisture in the GMM was investigated on the mechanical strength of the MN 14 by tuning crosslinking time. Increased crosslinking time leads to additional curing which results in less moisture released from the GMM, and less water released from the gel reduces degradation of the MN shell 20. Five different MNs 14 were prepared: d-HMND patch 10 without GMM (only PLGA shell 20) and d-HMND patch 10 with GMM crosslinked for 1, 2, 3 and 4 minutes respectively. Then, mechanical strength of the d-HMND patches 10 were measured after 24 hours using a custom force measurement system. A metal pillar with a flat surface gently pressed down a patch 10 fixed on top of a load-cell at a controlled speed of 1.8 μm/s. The reaction force was recorded in real time over the displacement of the metal pillar. The first peak in the force-response curve indicates the yield strength of the microneedles 14 of the patch 10 (inset of FIG. 5A). The mechanical strength of the microneedles 14 of the patch 10 decreased by as the degree of crosslinking was reduced (FIG. 5A). Lower degrees of crosslinking reduced the GMM's ability to trap moisture which caused more moisture outside of the GMM to degrade the microneedles 14. The microneedles 14 crosslinked for 1 and 2 minutes showed a statistically significant decrease in mechanical integrity relative to the exclusively PLGA shell 20 (control group). Conversely, there was not a statistically significant change in failure force when comparing microneedles 14 crosslinked for 3 and 4 minutes. Further study showed that average failure force was higher for samples crosslinked for 4 minutes as opposed to 3 minutes. Additionally, the properties of the microneedles 14 cured for 4 minutes closely matched the rigidity of the patch 10 composed of only a PLGA shell 20 (FIG. 5B). Beyond the mechanical properties of the patch 10, it was also observed that MSC viability was greater than 90% at crosslinking times between 3 and 4 minutes (FIG. 3A). Therefore, in order to preserve the mechanical strength and cell viability, the GMM curing time was selected to be 4 minutes.

After optimizing the fabrication, the insertion ability of the microneedles 14 was tested using ex vivo mouse skin 110. The ex vivo tissue 110 was harvested and used within 4 hours. Once again, to visualize the microneedles 14 within the tissue, RB was used. After patching, the treated tissue 110 was frozen and cryosectioned. Immediately before the patch 10 was applied to the skin 110, the surface of the tissue 110 was wetted with saline. After applying the patch 10 to the tissue 110, the PLGA shells 20 were expected to slightly degrade via hydrolysis and the dismantlement of the microneedles 14 would proceed. The application time of the MNs 14 was optimized to balance successful detachment from the base substrate layer 12 and the minimization of patching time. After studying an array of different contact times, only those in excess of 1 minute resulted in the detachment and proper placement of greater than 90% of MNs 14 (data not shown). It was found that the MNs 14 were successfully transferred from the flexible base substrate layer 12 to the target tissue 110 and the microneedles 14 penetrated deep enough into the tissue 110 to facilitate localized MSC 100 delivery (FIG. 5C).

MSCs in Microneedles (d-HMND)

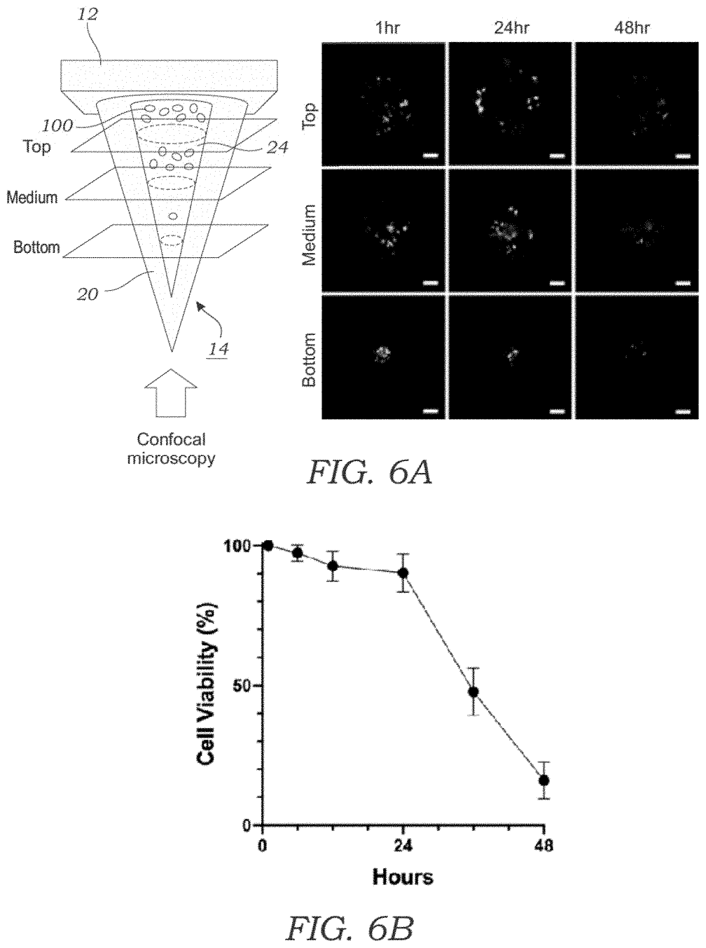

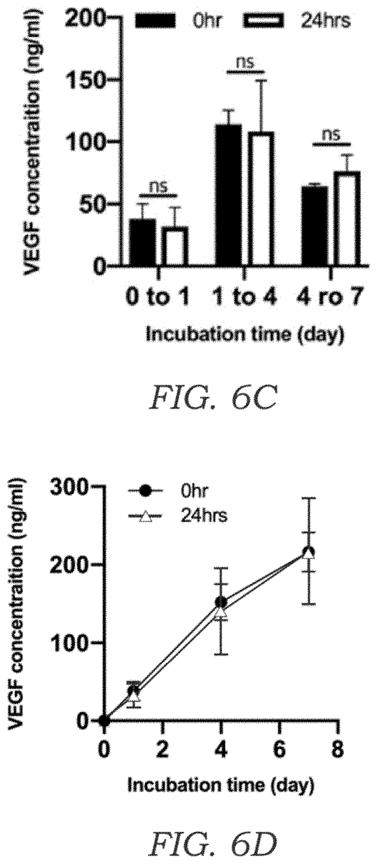

Despite effective manufacturing protocols and ideal mechanical properties for tissue penetration, the application of regenerative therapies using microneedles cannot be realized without viable, functional MSCs 100 encapsulated in the microneedles 14. To demonstrate the systems aptitude for the delivery of live cells 100, the viability of MSCs 100 in the MINI loaded in the PLGA shell 20 was investigated. Although the PLGA shell 20 initially inhibits the diffusion of nutrients to sustain the cells 100, the internal GMM is based on nutrient-rich media that can to sustain the MSCs 100 for a certain period of time. Patches 10 were prepared and stored them for up to 48 hours in an incubator at 36.5° C. The microneedles 14 were deconstructed and the base substrate layer was removed to assay MSC viability at 1, 6, 12, 24, 36 and 48-hour time-points after patch 10 fabrication. The microneedle structure was divided into three equal length segments (top, middle, and bottom), as shown in FIG. 6A, to better observe regional MSC viability. The results were also averaged to provide a representative viability for the entire structure. As hypothesized, cell viability remained above 90% up to 24 hours after production (FIG. 6B); however, later time points showed a sharp decrease with approximately 10% viability after 48 hours. This experiment indicated that the patch 10 should be used earlier than 24 hours after fabrication. Future animal experiments used patches 10 formed within 12-24 hours after production.

Even though the cell viability is maintained above 90% for 24 hours within the PLGA shell 20, it is also essential that the MSCs 100 function normally to enhance their regenerative effect. To verify this behavior, the paracrine signaling of MSCs 100 was investigated. To study the secretion of pro-angiogenic molecules, vascular endothelial growth factor (VEGF) was selected as a representative biomarker promoting angiogenesis, as it is a well-known mitogen for endothelial cells. VEGF was quantified by assaying the conditioned media from MSCs 100 cultured in two different disks of GMM (2 mm height×8 mm diameter). One sample was stored in cell media immediately after GMM production (0 hr group), and the other was stored in cell media after being stored for 24 hours covered with a PDMS mold and PLGA film after MINI production (24 hrs group). The conditioned media was collected at days 1, 4 and 7 and performed an ELISA. MSCs 100 in both samples secreted VEGF throughout the study showing the highest secretion between days 1 and 4 (FIG. 6C). Interestingly, it was found no statistically significant difference in VEGF secretion between the two groups at all time points. This indicates that the MSCs 100 in the microneedles 14 can maintain both their viability and functionality for up to 24 hours post-fabrication. Both groups also demonstrated stable VEGF secretion over one week (FIG. 6D).

Therapeutic Effect of Microneedles (d-HMND)

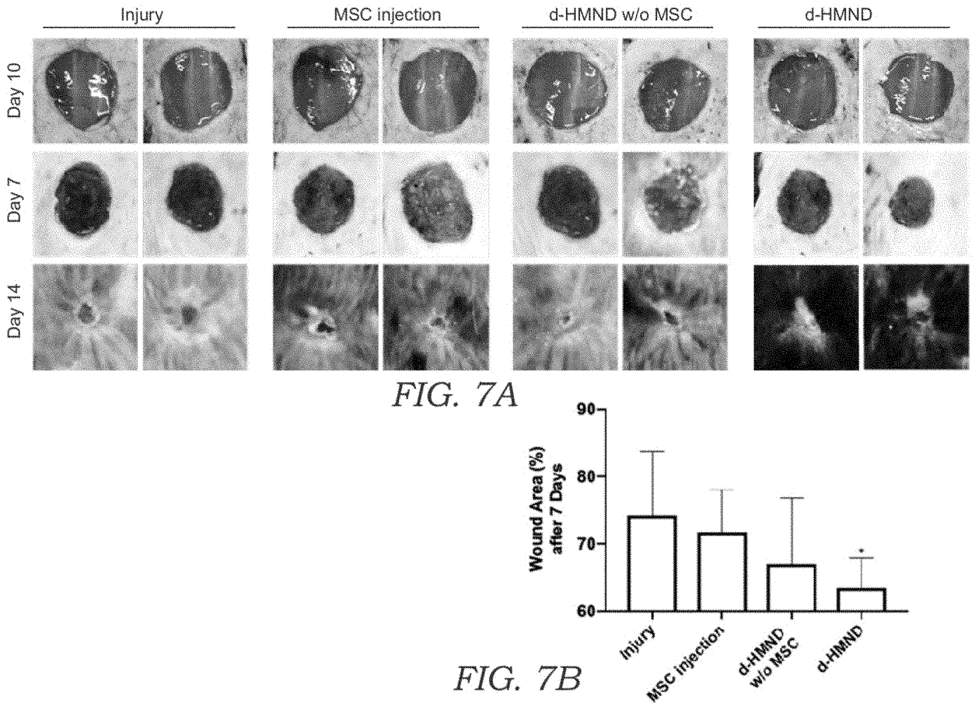

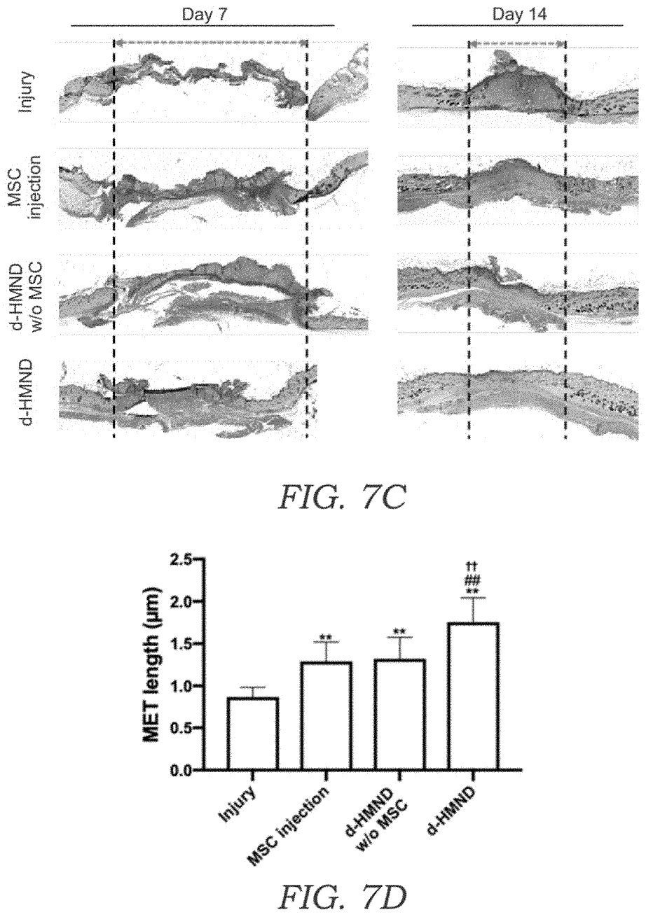

The excisional wound model is one of the most commonly employed mouse models for studying general regeneration processes. A full skin thickness excisional wound extending through the panniculus carnosus was created on the dorsum to make injury group (no treatment). Then MSC injection, patches 10 (i.e., d-HMND) with and without MSCs 100 (d-HMND group and d-HMND w/o MSC group, respectively) groups were prepared. The MSC injection group was intradermally injected with 0.1 mL of PBS seeded with 10⁶ MSCs near the wound site. Relative to the number of cells delivered by the 8×8 d-HMND array as described herein, 200 times more cells were injected into the wound. As for the patches 10 used in the in vivo experiments, the volume of each microneedle 14 was approximately 7×10⁻³ μL and contained seventy (70) cells 100, assuming the density of the prepolymer solution with MSCs is 1 g/mL. Resultingly, the 8×8 microneedle array delivered approximately 4500 cells in total. The d-HMND group was studied by comparing with the d-HMND w/o MSC group as a control. Wounds were photographed at Day 7 and 14, and wound closure was determined based on wound size relative to the original wound dimensions (FIGS. 7A, 7B). Wound healing rates were calculated 7 and 14 days after injury. The rates were 74.2±9.5% and 11.4±0.5%, respectively, in the injury group; 71.7±6.3% and 11.5±3.6% in the MSC injection group; 67.0±9.8% and 10.9±4.0% in the d-HMND w/o MSC group; and 63.4±4.4% and 7.3±4.5% in the d-HMND group. The findings suggested that the wound healing rate was slowed at both time points with treatment from the d-HMND. After 14 days there was no statistically significant difference in wound area amongst the various groups, however the untreated injury group was found to have the slowest wound contraction over the entire period. Among the groups receiving treatment, the d-HMND group and the MSC-treated groups exhibited similar wound healing patterns. Wound contraction was most rapid in animals treated with the MSC-loaded d-HMND. Additionally, mice that did not receive treatment did not show hair regeneration around the wound, whereas in all treated groups (MSC, d-HMND w/o MSC and d-HMND) hair regrew as the wound closed. Interestingly, animals treated with the MSC-loaded d-HMND group showed increased hair regeneration, even compared to other treated groups.

The wound healing effect of the microneedle-containing patches 10 was further analyzed through histological evaluation (FIG. 7C). Low-magnification histological analysis revealed that treatment with the d-HMND improved connectivity (tissue migration) in the wound bed compared to other groups. After 14 days, inflammation and granulation tissue persisted in the untreated group, whereas the treated groups (MSC, d-HMND w/o MSC and d-HMND) all showed granulation tissue maturation. In particular, the d-HMND group showed evidence of transition to the remodeling phase of wound healing, indicated by a decrease in the thickness of the fibrotic tissue and an increase in the number of hair follicles around the wound. Re-epithelialization of the wound bed was measured in cross-sectioned tissue on day 7 (FIG. 7D). The d-HMND group demonstrated significantly more re-epithelialization (48.1±12.9%, n=5) than the other groups (injury; 21.0±2.9%, MSC injection; 35.6±8.7% and d-HMND w/o MSC; 35.5±5.7%). Furthermore, the d-HMND group enhanced keratinocyte migration towards the center of the wound leading to increased migrating epidermal tongue (MET) length relative to the other groups (FIG. 7E). Based upon the various tissue analyses conducted, the in vivo experiments validate the findings of the in vitro experiments and confirm that the patches 10 can be used to improve skin wound healing through the delivery of MSCs 100.

A cell delivery device in the form of a patch 10 with a removable base substrate layer 12 that deposits a plurality of microneedles 14 in tissue 110 has been described that can be used, in one embodiment, for enhancing wound healing. It uses an array of microneedles 14 to facilitate localized MSC 100 delivery with a minimal dose of cells 100. A biodegradable PLGA shell 20 was formed by a two-step molding process using plasma surface modification. GMM with a 30 kPa compressive modulus was characterized and optimized for MSC viability. GMM was loaded into the PLGA shells 20 and the microneedles 14 were transferred to commercial double-sided tape which was used as the base substrate layer 12. The array of microneedles 14 showed excellent mechanical integrity with strength sufficient to penetrate the target tissue 110. The microneedles 14 were able to be separated from the base substrate layer 12 substrate after application to the target tissue 110; this delivery mechanism ensures that the base substrate layer 12 does not elicit a foreign body response to as the microneedles 14 composed of PLGA, MSCs 100, and a biodegradable GelMA 24 scaffold have all been shown to be biocompatible. The use of cell media to make the GMM ensured that the nutrient supply was sufficient to maintain cell viability above 90% for 24 hours after device production. VEGF secretion, an indicator of the regenerative potential of the stem cells, by the MSCs 100 in the closed system (no nutrient supply from outside) was also demonstrated. MSCs 100 loaded in GMM and surrounded by PDMS and PLGA for 24 hours, showed VEGF secretion profiles similar to that of cultured MSCs immediately after fabrication. Lastly, a full-thickness skin excisional wound mouse model was used to test the in vivo therapeutic efficacy of the delivered microneedles 14. Animals treated with the patches 10 showed elevated wound closure rates and improved re-epithelialization compared to the controls. The patches 10 are an innovative cell therapy delivery technology that shows great promise for improved treatment of skin wounds using MSCs 100.

Synthesis of GelMA: 10 g of Gelatin from porcine skin (G1890, Sigma Aldrich, USA) was dissolved in 100 mL of PBS at 50° C. for 1 hour. Next, 8 mL of methacrylic anhydride (276685, Sigma Aldrich, USA) was added using a burette to the gelatin solution and stirred at 50° C. for 2 hours before 100 mL of PBS was added and mixed at 50° C. Subsequently, the solution was dialyzed for 5 days at 40° C. using dialysis bag (888-11530, Spectrum Chemical Mfg. Corp., USA) to remove impurities. The processed solution was filtered by a vacuum filtration cup with 0.22 μm pores and then was frozen at −80° C. The frozen GelMA was lyophilized for 3 days.

MSC Culture: Human MSCs from bone marrow were purchased from ATCC (Cat. No. PCS-500-012) and cultured in DMEM-LG (Sigma Aldrich, USA) supplemented with 10% FBS (Sigma Aldrich, USA) and 1% PS (Invitrogen, USA). Cells were passaged or collected at approximately 80% confluency. When passaging, cells were rinsed 3 times with PBS and trypsin-EDTA (Gibco, USA) solution was added. When the cells were detached from the flask, an equal volume of cell culture medium was added to quench the trypsin. Dissociated cells were transferred to centrifuge tube for further use. Passage 3˜5 MSCs were used in the experiments.

Fabrication of cell delivery device (e.g., patches or d-HMND): To obtain the microneedle structures, lithography, multiple layer deposition, and wet etching processes were used. To alter the size and shape of the microneedles, the size of the square micropattern (1,750×1,750 μm) on the photomask was manipulated. At first, oxide and a nitride layers were deposited on a 4-inch wafer by furnace and low-pressure chemical vapor deposition, in sequence. An SU-8 layer was then spread by spin coating and exposed to UV light through a lithographic film photomask bearing an array of squares. After photoresist ashing, the exposed Si₃N₄ and SiO₂ layers were removed by reactive ion etching to leave a micropattern of square islands. The patterned wafer was dipped in 29% KOH solution at 79° C. and washed in a bubbling water bath. Finally, an octagonal cone-shaped, 700 μm length male MN array was fabricated with a 1.5 aspect ratio. Subsequently, a polydimethylsiloxane (PDMS) mold 40 containing an array of MN cavities 42 was prepared by casting the PDMS over the male MNs. To make the PLGA shell 20 of the MN 14, PLGA solution (PLGA:DMSO=1:3, w/w) was cast into the mold 40 multiple times. Prior to casting, the surface of the PDMS mold 40 was modified by O₂ plasma to make the surface hydrophilic and allow the PLGA solution to fill the cavity 42 by adhering to the walls of the PDMS cavity 42. PLGA was cast over the mold 40 and evaporated under vacuum to solidify the PLGA shell 20 and remove bubbles. The same process was repeated to thicken and reinforce the PLGA shell 20. Once the final shell 20 was formed, GMM was cast over the mold 40 containing the PLGA shells 20 and covered by the double-sided tape as the base substrate layer 12 to yield the assembled patch 10 or d-HMND.

Mechanical testing of GelMA and d-HMND: Compressive tests to measure the stiffness of the hydrogels were conducted using a universal testing machine (Instron 5524, Instron, USA) with a 10 N load cell. To make the samples for mechanical testing, a standardized gel structure crosslinked with different UV exposure times was used to vary the mechanical properties. 14 mW/cm² UV light was used. The compressive modulus of gels crosslinked for between 2 and 5 minutes was found. In parallel, MSC viability was investigated in the gels with varying stiffness. Cell viability was evaluated with a LIVE/DEAD Viability/Cytotoxicity Kit. A solution consisting of 0.05% Calcein and 0.2% ethidium homodimer-1 in PBS was added to each well and incubated for 30 minutes. After three washes with PBS the samples were imaged with a fluorescent microscope to find the optimal GelMA property. Because the d-HMND is a closed system that does not provide additional nutrients once manufactured, it was feared that MSC viability would decrease rapidly with time. Several d-HMNDs were prepared and cell viability was quantified at different time points up to 3 days.

Cell viability test and ELISA assay: A live/dead assay was performed based on the vendor's instructions. Briefly, cells in GelMA were washed twice with PBS and incubated in Calcein AM/ethidium homodimer-1 staining solution (described above) for 30 minutes. After incubation, the cells were washed three times with PBS before fluorescent imaging. The human VEGF sandwich assay ELISA (Sigma-Aldrich) was performed based on the supplier's instructions. Briefly, 100 μl of each standard and sample were pipetted into wells coated with capture antibodies and placed in an incubator for 2.5 hours. In succession, 100 μl of detection antibody were added to each well and left for 1 hour, then 100 μl of streptavidin for 45 min, 100 μl of substrate solution for 20 min, and 50 μl of stop solution to halt the reactions. In between each step, the wells were washed four times with wash buffer. Using a plate reader (BIOTEK Fluorescent plate reader, Synergy HTX multimode reader), absorbance of 450 nm light was used to assay each well.

In vivo therapeutic test: All animal experiments were approved by the UCLA Animal Research Committee (UCLA ARC #2018-003-01E). All animals were treated in compliance with the National Research Council criteria as outlined in the “Guide for the Care of Laboratory Animals” prepared by the Institute of Laboratory Animal Resources and published by the National Institute of Health. Forty 7-week-old, C57BL/6J male mice (average weight: 20 grams) were purchased from Jackson Laboratory (Sacramento, Calif., USA). Full-thickness excisional wounds were made on anesthetized mice (gas anesthesia, 1.5% isoflurane in 100% O₂). A circular incision was made on the middle line of the dorsal skin using a 10-mm biopsy punch (Miltex, York, Pa., USA). Full-thickness skin tissue, including the epidermis, dermis, subcutis, and muscularis, was separated by the blunt tip of a Metzenbaum scissors, and cut off along the incision. Mice were sacrificed for histology 7- and 14-days post-operation.

A total of 40 mice were used in the study and randomly divided into four groups. For the untreated injury group (n=5, control), 100 μL of PBS was applied onto the wound bed. Mice in the MSC injection group (n=5) were intradermally injected with 100 μL of PBS containing 10⁷ cells per mL at four injection sites around the wound. Another group of mice had d-HMNDs without MSCs applied (n=5), 8×8 d-HMND arrays without MSCs were applied directly to the wound bed. Similarly, the d-HMND group (n=5) had 8×8 d-HMND arrays containing MSCs also applied directly to the wound bed.

Histology and wound healing evaluation: Digital photographs of each wound was taken at days 0, 7, and 14. Wound closure time was defined as when the wound bed was re-epithelialized and completely filled with new tissue. Wound area was defined by tracing the margin of wound and measured by ImageJ program (National Institute of Health, USA). The investigators who measured the wound were blinded. Wound healing rate was calculated as follows: (Area_{original wound}−Area_{remaining wound})/Area_{original wound}×100. Skin specimens taken from the wound and the surrounding unwounded area were collected and fixed in 10% neutral buffered formalin (Leica Biosystems, IL, USA). This was followed by a general procedure for histological analysis and embedded in paraffin. 4 μm sections were processed with routine hematoxylin (Leica Biosystems) and eosin (Sigma) (HE) stain. Histology images were acquired on a Nikon inverted microscope. Quantitative data, such as re-epithelialization % and MET length, was measured using the AmScope image analysis software (AmScope, Irvine, Calif., USA). The re-epithelialization ratio (%) was measured in the HE-stained sections on day 7 (n=5/group). The width of the wound and distance covered by newly formed epithelium was measured and the re-epithelialization percentage was calculated by the following formula: % re-epithelialization=(distance covered by the epithelium/width of wound bed)×100%. All histology results described herein were expressed as mean±SD and statistical analysis was performed using GraphPad Instat (Graphpad software Inc., La Jolla, Calif., USA). The statistical significance of differences was assessed by one-way ANOVA and Bonferroni post-hoc paired comparisons tests. P-values less than 0.05 between experimental groups were considered statistically significant.

While embodiments of the present invention have been shown and described, various modifications may be made without departing from the scope of the present invention. For example, the hardened shell 20 may be made from a variety of different materials with other cytocompatible hydrogel materials 24 beyond those specifically identified in the experimental results herein. Likewise, a variety of cells 100 may be loaded into the microneedles 14. The invention, therefore, should not be limited, except to the following claims, and their equivalents.

1. A delivery device for the localized delivery of live cells to living tissue comprising:
a base substrate layer having a plurality of microneedles extending away from the surface of the base substrate layer, wherein the plurality of microneedles are formed from an outer hardened shell comprising a biodegradable or dissolvable polymer and an inner region containing a cytocompatible hydrogel-based material mixed with the live cells, and wherein the plurality of microneedles are detachable from the base substrate layer.

2. The delivery device of claim 1, wherein the plurality of microneedles comprise sharpened tips.

3. The delivery device of claim 1, wherein the outer hardened shell of the plurality of microneedles is formed from a material selected from the group comprising: poly(lactic-co-glycolic) acid (PLGA), polyvinylpyrrolidone (PVP), polyvinyl alcohol (PVA), polycaprolactone (PCL), chitosan, polyhydroxyalkanoates (PHA), polyanhydrides, polyvalerolactone, polydioxanone, polyurethane (PUR), or polyphosphazenes.

4. The delivery device of claim 1, wherein the cytocompatible hydrogel-based material is selected from the group comprising: crosslinked gelatin methacryloyl (GeIMA) hydrogel, silk fibroin hydrogel, collagen-based hydrogel, alginate-based hydrogel, hyaluronic acid-based hydrogel, cellulose-based hydrogel, or poly(ethylene glycol) (PEG)-based hydrogel.

5. The delivery device of claim 1, wherein the plurality of microneedles have a length of less than about 1.5 mm.

6. The delivery device of claim 1, wherein the plurality of microneedles have a length within the range of about 10 μm to about 1,500 μm.

7. The delivery device of claim 1, wherein the base substrate layer comprises a flexible substrate having an adhesive material or glue disposed thereon.

8. The delivery device of claim 7, wherein the base substrate layer comprises an adhesive layer.

9. The delivery device of claim 1, wherein the plurality of microneedles are disposed in an array on the base substrate layer.

10. A method of treating tissue comprising:
inserting a plurality of microneedles into tissue, wherein the plurality of microneedles are formed from an outer hardened shell comprising a biodegradable or dissolvable polymer and an inner region containing a cytocompatible hydrogel-based material mixed with the live cells.

11. The method of treating tissue of claim 10, wherein the plurality of microneedles are temporarily adhered to a base substrate layer that detaches from the plurality of microneedles and is removed after insertion of the plurality of microneedles into the tissue.

12. The method of treating tissue of claim 10, wherein the outer hardened shell of the plurality of microneedles are formed from a material selected from the group comprising: poly(lactic-co-glycolic) acid (PLGA), polyvinylpyrrolidone (PVP), polyvinyl alcohol (PVA), polycaprolactone (PCL), chitosan, polyhydroxyalkanoates (PHA), polyanhydrides, polyvalerolactone, polydioxanone, polyurethane (PUR), or polyphosphazenes.

13. The method of treating tissue of claim 10, wherein the cytocompatible hydrogel-based material is selected from the group comprising: crosslinked gelatin methacryloyl (GeIMA) hydrogel, silk fibroin hydrogel, collagen-based hydrogel, alginate-based hydrogel, hyaluronic acid-based hydrogel, cellulose-based hydrogel, or poly(ethylene glycol) (PEG)-based hydrogel.

14. The method of claim 11, wherein the base substrate layer is flexible.

15. A method of using the delivery device of claim 1 comprising:
placing the delivery device on living tissue of a mammal such that the plurality of microneedles penetrates into the tissue; and
removing the base substrate layer from contact with the tissue, wherein the plurality of microneedles separate from the base substrate layer and remain in the tissue.

16. The method of using the delivery device of claim 15, wherein the base substrate layer is removed after at least one (1) minute has elapsed.

17. The method of using the delivery device of claim 15, wherein a majority of the microneedles of the delivery device separate from the base substrate layer and remain in the tissue upon removal of the base substrate layer.

18. A method of manufacturing a delivery device for the localized delivery of live cells to living tissue comprising:
providing a mold containing a plurality of needle-shaped cavities therein;
applying a solution containing poly(lactic-co-glycolic) acid (PLGA) in a solvent on the mold allowing the solvent to evaporate to form shells in the needle shaped cavities;
applying a solution of gelatin methacryloyl (GeIMA), live cells, and a photoinitiator on the mold containing the shells;
irradiating the mold containing the solution with light to crosslink the GeIMA to form the plurality of microneedles;
applying a base substrate layer having an adhesive thereon to the mold; and
removing the base substrate layer from the mold with the plurality of microneedles adhered thereto.

19. The method of claim 18, wherein the shells are formed by performing multiple rounds of applying the solution containing poly(lactic-co-glycolic) acid (PLGA) followed by solvent evaporation.

20. The method of claim 18, wherein the mold is irradiated with light for between 2 and 6 minutes.

21. (canceled)

22. A method of manufacturing a delivery device for the localized delivery of live cells to living tissue comprising:
providing a mold containing a plurality of needle-shaped cavities therein;
applying a solution containing a biodegradable or dissolvable polymer in a solvent on the mold and allowing the solvent to evaporate to form shells in the needle shaped cavities;
applying a solution of a cytocompatible hydrogel-based material, live cells, and a photoinitiator on the mold containing the shells;
irradiating the mold containing the solution with light to crosslink the hydrogel-based material to form the plurality of microneedles;
applying a base substrate layer having an adhesive thereon to the mold; and
removing the base substrate layer from the mold with the plurality of microneedles adhered thereto.
